(19) United States
(12) Patent Application Publication
(10) Pub. No.: US 2014/0350120 A1
(43) Pub. Date: Nov. 27, 2014

(54) TOPICAL COMPOSITION COMPRISING AN INGENOL DERIVATIVE AND AN OILY SOLVENT

(71) Applicant: LEO LABORATORIES LIMITED, Dublin (IE)

(72) Inventors: Per Ola Arvidsson, Ballerup (DK); Edit Farkas, Ballerup (DK); Karsten Petersson, Ballerup (DK); Gert Hoy, Ballerup (DK)

(73) Assignee: Leo Laboratories Limited, Dublin (IE)

(21) Appl. No.: 14/364,567

(22) PCT Filed: Dec. 12, 2012
(86) PCT No.: PCT/IB2012/057250

§ 371 Date: Jun. 11, 2014

Related U.S. Application Data
(60) Provisional application No. 61/569,643, filed on Dec. 12, 2011.

(51) Int. Cl. A61K 47/14; A61K 47/10; A61K 47/08; A61K 31/122

(52) U.S. Cl. CPC A61K 47/14; A61K 31/122; A61K 47/10; A61K 47/08
USPC 514/691

(57) ABSTRACT

A substantially anhydrous topical composition comprises a homogenous mixture of (a) a therapeutically effective amount of an ingenol derivative in dissolved form; (b) an oily solvent for the ingenol derivative, (c) an acidic compound, and (d) optionally a pharmaceutically acceptable non-solvent lipid carrier.

### FIELD OF INVENTION

The present invention relates to a topical pharmaceutical formulation comprising an ingenol derivative as a pharmacologically active agent and an oily solvent for the ingenol derivative.

### BACKGROUND OF THE INVENTION

The invention provides a pharmaceutical formulation suitable for topical application of the compound ingenol-3-angelate (2-methyl-2(Z)-butenoic acid (1aR,2S,5R,5aS,6S,8aS,9R,10aR)-5,5a-dihydroxy-4-(hydroxymethyl)-1,1,7,9-tetramethyl-11-oxo-1a,2,5,5a,6,9,10,10a-octahydro-1H-2,8a-methanocyclopenta[a]cyclopropa[e]cyclodecen-6-yl ester; PEP005). Ingenol-3-angelate (PEP005) is a protein kinase C activator in phase III clinical development for the treatment of actinic keratosis. The drug candidate is also in phase II trials for non-melanoma skin cancer [Ogbourne, S. M.; Anti-cancer Drugs, (2007), 18, 357-62].

The compound ingenol-3-angelate (PEP005) [Sayed, M. D. et. al.; Experienta, (1980), 36, 1206-1207] can be isolated from various Euphorbia species, and particularly from Euphorbia peplus [Hohmann, J. et. al; Planta Med., (2000), 66, 291-294] and Euphorbia drummondii by extraction followed by chromatography as described in U.S. Pat. No. 7,449,492. Pharmaceutical formulation of the compound has been described in WO200768963.

Angelic acid and angelic acid esters such as ingenol-3-angelate, are prone to isomerisation of the double bond to form the tiglate ester, particularly at basic pH or when subjected to heat [Beeby, P., Tetrahedron Lett. (1977), 38, 3379-3382, Hoskins, W. M., J. Chem. Soc. Perkin Trans. 1, (1977), 538-544, Bohlmann, F. et. al., Chem. Ber. (1970), 103, 561-563]. As a consequence only carefully optimised conditions for ester formation can be applied in the synthetic preparation of ingenol-3-angelate.

Furthermore, ingenol-3-acylates are known to be unstable as they rearrange to afford the ingenol-5-acylates and ingenol-20-acylates [Sorg, B. et. al, Z. Naturforsch., (1982), 37B, 748-756].

WO 2007/068963 discloses an aqueous gel formulation for the treatment of skin cancer in which ingenol angelate is dissolved in an aprotic solvent, the formulation further comprising an acidifying agent such that the pH of the formulation is no greater than 4.5. The aqueous gel is generally stored at refrigeration temperature.

One object of the invention is therefore to provide a composition of the ingenol derivative which is stable at room temperature for the entire shelf-life of the composition.

Another object of the invention is to provide a composition exhibiting favourable penetration characteristics and biological activity.

A further object of the invention is to provide a composition with reduced skin irritation and favourable cosmetic properties and improved patient compliance.

### SUMMARY OF THE INVENTION

In the research leading to the present invention, it was an object to identify a solvent which is as effective at dissolving compounds such as ingenol derivatives as low-molecular alcohols or diols when used on their own as co-solvents in admixture with an aqueous phase. It has surprisingly been found that ingenol derivatives are dissolved in certain oily solvents to the same extent as in the alcohol-water solvent system disclosed in WO 2007/068963.

In one aspect, the invention relates to a substantially anhydrous topical composition comprising a homogenous mixture of

(a) a therapeutically effective amount of an ingenol derivative in dissolved form;
(b) an oily solvent for the ingenol derivative,
(c) an acidic compound, and
(d) optionally a pharmaceutically acceptable non-solvent lipid carrier.

Ingenol derivatives such as ingenol-3-angelate are known to be extremely sensitive to higher pH conditions (pH above about 4.5 in aqueous compositions or alkaline reacting substances in non-aqueous compositions) which contribute to the isomerization of the angelic acid ester to the tiglate ester and the acyl migration of the angelic acid moiety. To ensure an adequate chemical stability of the substance throughout the shelf-life of the composition, the composition should include an acidic compound capable of neutralizing alkaline impurities which may be present in one or more of the excipients of the composition and which are detrimental to the chemical stability of ingenol derivatives.

The present composition has been found to result in a satisfactory chemical stability of the ingenol derivative included therein permitting the composition to be stored at room temperature (about 25° C.) throughout its shelf-life. The satisfactory stability may partly be ascribed to the substantial absence of water in the composition, and partly to the presence of a compound capable of neutralizing any alkaline impurities that may be present in individual vehicle components.

Human skin, in particular the outer layer, the stratum corneum, provides an effective barrier against penetration of microbial pathogens and toxic chemicals. While this property of skin is generally beneficial, it complicates the dermal administration of pharmaceuticals in that a large quantity, if not most, of the active ingredient applied on the skin of a patient suffering from a dermal disease may not penetrate into the viable layers of the skin (the dermis and epidermis) where it exerts its activity. To ensure an adequate penetration of the active ingredient to the dermis and epidermis, it is generally preferred to include the active ingredient in a dissolved state, typically in the presence of a solvent in the form of an alcohol, e.g. ethanol or isopropanol. When used on their own as solvents, alcohols such as isopropanol and diols may give rise to significant skin irritation as they tend to dry out the skin. The drying out effect may, however, be mitigated by including an oily phase in the composition as the oil or oils may act as emollients and/or humectants. The composition may also be more easily spreadable when an oily phase is included as it evaporates less quickly than alcohol.

While in some embodiments, the present composition may include a lower alcohol as a cosolvent, it is generally present in a much lower amount than in the aqueous gel formulation disclosed in WO 2007/068963. Even so, the present composition has surprisingly been found to exhibit improved penetration of the ingenol derivative into the viable layers of the skin, but not higher permeation through the skin than seen with the hydrogel formulation disclosed in WO 2007/068963). It is possible that the improved penetration may result in a higher efficacy of composition of the invention.

In another aspect of the invention, the present composition may be used in the treatment of a dermal disease or condition.

### DETAILED DESCRIPTION OF THE INVENTION

### Definitions

In the present context, the term “ingenol derivative” is intended to mean an ingenol compound isolated from a species of Euphorbia, in particular from E. peplus or an ingenol derivative prepared by chemical synthesis or by a semi-synthetic route, e.g. as disclosed in copending application No. PCT/DK2011/000081. Examples of ingenol derivatives that may be included in the present compositions are ingenol-3-angelate, ingenol-5-angelate, ingenol-20-angelate, 20-O-acetyl-ingenol-3-angelate and 20-deoxy-ingenol-3-angelate. Ingenol-3-angelate, also known as ingenol-3-mebutate or PEP005, is currently in development for the treatment of actinic keratosis.

The term “storage stability” is intended to indicate that the composition exhibits chemical and physical stability characteristics that permit storage of the composition, at refrigeration or, preferably, room temperature for a sufficient period of time (the shelf-life of the composition) to make the composition commercially viable, such as at least 12 months, in particular at least 18 months, and preferably at least 2 years.

The term “chemical stability” or “chemically stable” is intended to indicate that no more than 10%, preferably no more than 6%, of the ingenol derivative degrades over the shelf-life of the product, typically 2 years. An approximation of chemical stability at room temperature is obtained by subjecting the composition to accelerated stability studies at 40° C. If less than about 3% of the substance, e.g. ingenol-3-angelate, has degraded after 3 months at 40° C. a shelf-life of 2 years at room temperature is considered obtainable.

The term “physical stability” or “physically stable” is intended to mean that the composition retains its macroscopic and microscopic appearance over the shelf-life of the product, e.g. that the ingenol derivative does not precipitate from the solvent phase or that there is no visible phase separation of the solvent phase and the carrier phase.

The term “skin penetration” is intended to mean the diffusion of the active ingredient into the different layers of the skin, i.e. the stratum corneum, epidermis and dermis.

The term “skin permeation” is intended to mean the flux of the active ingredient through the skin into the systemic circulation or, in case of in vitro studies, the receptor fluid of the Franz cell apparatus used in the experiment.

The term “medium chain triglycerides” is intended to indicate triglyceride esters of fatty acids with a chain length of 6-12 carbon atoms. A currently favoured example of medium chain triglycerides is a mixture of caprylic (C₈) and capric (C₁₀) triglycerides, e.g. available under the trade name Miglyol 812.

The term “substantially anhydrous” is intended to mean that the content of free water in the composition is less than about 2% by weight, preferably less than about 1% by weight, such as less than about 0.5% by weight, of the composition.

The term “acidic compound” is intended to indicate a compound capable of providing a net overall acidic environment in the composition and/or capable of neutralizing alkaline impurities detrimental to the chemical stability of the ingenol derivative.

The term “occlusive agent” is intended to indicate a lipid substance that forms a layer on the surface of the skin on application of the composition. The lipid layer forms a hydration barrier sufficient to result in reduction of transepidermal water loss, resulting in skin hydration.

### Embodiments

The oily solvent for the ingenol derivative included in the present composition may be selected from a vegetable oil, e.g. sesame oil, sunflower oil, palm kernel oil, corn oil, safflower oil, olive oil, avocado oil, jojoba oil, almond oil, canola oil, coconut oil, cottonseed oil, peanut oil, soybean oil, wheat germ oil or grape kernel oil, a highly purified vegetable oil, e.g. medium chain triglycerides, long chain triglycerides, castor oil, caprylic/capric mono- and diglycerides or caprylic/capric mono-, di- and triglycerides, a synthetic oil such as fatty alcohol esters of C₁₀₋₁₈ alkanoic acids, e.g. isopropyl myristate, isopropyl palmitate, isopropyl linoleate, isopropyl monooleate or isostearyl isostearate, polyoxypropylene fatty alkyl ethers such as polyoxypropylene-15-stearyl ether, an alkyl or dialkyl ester such as ethyl oleate, diisopropyl adipate or cetearyl octanoate, a mono- or diglyceride such as glyceryl monooleate, or a fatty alcohol such as oleyl alcohol, or mixtures thereof.

The oily solvent is included in the composition in an amount in the range of from about 1% to about 98% by weight of the composition, preferably about 2.5-75% by weight, about 5-50% by weight or about 5-40% by weight, such as about 5% or about 10% or about 15% or about 20% or about 25% or about 30% by weight of the composition.

The composition may further comprise a co-solvent selected from the group consisting of lower alcohols, such as n-propanol, isopropanol, n-butanol, 2-butanol or benzyl alcohol, or diols such as propylene glycol. This type of co-solvent may act as a penetration enhancer aiding the penetration of the ingenol derivative into the skin. Alternative penetration enhancers may be selected from propylene carbonate, Transcutol, a pyrrolidone such as N-methylpyrrolidone or N-hydroxyalkylpyrrolidone, an azone, menthol, eucalyptol, nicotinamide, glycerol, mono-di- or polyglycols, ethylacetate or Eugenol. Addition of a co-solvent may result in an improved physical stability of the composition.

The co-solvent/penetration enhancer may be present in an amount of 0.5-20%, such as 1-15%, e.g. 1%, 1.5%, 2%, 2.5% 3% or 5% by weight of the composition.

The composition may further include a non-solvent lipid carrier which acts as an occlusive agent. The lipid carrier may be selected from a mineral oil, e.g. liquid paraffin, or a hydrocarbon or mixture of hydrocarbons with chain lengths ranging from C₅ to C₆₀. A frequently used lipid carrier is petrolatum, or white soft paraffin, which is composed of hydrocarbons of different chain lengths peaking at about C₄₀₋₄₄, or a mixture of petrolatum and liquid paraffin (consisting of hydrocarbons of different chain lengths peaking at C₂₈₋₄₀). While petrolatum provides occlusion of the treated skin surface, reducing transdermal loss of water and potentiating the therapeutic effect of the active ingredient in the composition, it tends to have a greasy and/or tacky feel which persists for quite some time after application, and it is not easily spreadable. It may therefore be preferred to employ paraffins consisting of hydrocarbons of a somewhat lower chain length, such as paraffins consisting of hydrocarbons with chain lengths peaking at C₁₄₋₁₆, C₁₈₋₂₂, C₂₀₋₂₂, C₂₀₋₂₆ or mixtures thereof (the hydrocarbon composition of the paraffins has been determined by gas chromatography). It has been found that such paraffins are more cosmetically acceptable in that they are less tacky and/or greasy on application and more easily spreadable. They are therefore expected to result in improved patient compliance. Suitable paraffins of this type, termed petrolatum jelly, are manufactured by Sonneborn and marketed under the trade name Sonnecone, e.g. Sonnecone CM, Sonnecone DM1, Sonnecone DM2 and Sonnecone HV. These paraffins are further disclosed and characterized in WO 2008/141078 which is incorporated herein by reference. The non-solvent lipid carrier may also be an iso-paraffin such as isohexadecane or squalane, or a silicone oil, e.g. cyclomethicone or dimethicone.

The occlusive agent may be included in an amount of from about 40% to about 90% by weight of the composition.

To impart a desired viscosity to the present composition, it may suitably include a lipophilic viscosity-increasing ingredient such as a wax. The wax may be a mineral wax composed of a mixture of high molecular weight hydrocarbons, e.g. saturated C₃₅₋₇₀ alkanes, such as microcrystalline wax. Alternatively, the wax may be a vegetable or animal wax, e.g. esters of C₁₄₋₃₂ fatty acids and C₁₄₋₃₂ fatty alcohols, such as beeswax or hydrogenated castor oil. Alternatively, the viscosity-increasing ingredient may be an inorganic substance such as fumed silica, e.g. available under the trade name Aerosil. The viscosity-increasing ingredient may also be selected from magnesium stearate, aluminium stearate, a sterol such as cholesterol, a long-chain saturated fatty alcohol such as cetostearyl alcohol or a silicone rubber or wax such as Dow Corning ST-Elastomer 10 or Dow Corning Silky Wax. The amount of viscosity-increasing ingredient may vary according to the viscosifying power of the ingredient, but may typically be in the range of about 0.5-20% by weight of the composition. When the viscosity-increasing ingredient is microcrystalline wax it is typically present in an amount in the range of about 0.5-30% by weight, of the composition. The viscosity-increasing ingredient included in the composition may also be a mixture of acrylamide acryloyldimethyl taurate copolymer, isohexadecane and polysorbate 80, e.g. available under the trade name SEPINEO P600, which may be included in an amount of about 1-10% by weight, such as about 2.5% by weight, of the composition, or alkylpolyglucoide, e.g. available under the trade name SEPINEO SE68, which may be included in an amount of 2-30% by weight, such as 5% by weight, of the composition.

The acidic compound included in the present composition may be selected from, e.g., fumed silica, which may be included in the composition in an amount of about 3-10% by weight such as about 5-9% by weight. Alternatively, neutralization of alkaline reacting substances may be provided by addition of a fatty acid such as oleic acid, linoleic acid, stearic acid, lauric acid, palmitic acid, capric acid, caprylic acid, pelargonic acid, adipic acid, sebacic acid or enanthic acid to the composition. The fatty acid may be included in an amount of about 0.5-5% by weight of the composition.

Examples of ingenol derivatives that may be included in the present composition are ingenol-3-angelate, ingenol-5-angelate, ingenol-20-angelate, 20-O-acetyl-ingenol-3-angelate and 20-deoxy-ingenol-3-angelate. A currently favoured ingenol derivative is ingenol-3-angelate, also known as ingenol-3-mebutate or PEP 005. The ingenol derivative may be included in the composition in an amount of about 0.001-0.5% by weight of the composition.

The composition of the invention may be used in the topical treatment of a dermal disease or condition. Examples of dermal diseases and conditions are actinic keratosis, seborrheic keratosis, skin cancer, such as basal cell carcinoma or squamous cell carcinoma, warts, keloids, scars, photoaged or photodamaged skin, or acne.

The term “skin cancer” is intended to include non-melanoma skin cancer, malignant melanoma, Merkel cell carcinoma, squamous cell carcinoma or basal cell carcinoma. Basal cell carcinomas include superficial basal cell carcinoma as well as nodular basal cell carcinoma.

The term “photodamaged skin” is intended to include cover fine lines, wrinkles and UV-ageing. UV ageing is often manifested by an increase in the epidermal thickness or epidermal atrophy and most notably by solar elastosis, the accumulation of elastin containing material just below the dermal-epidermal junction. Collagen and elastic fibres become fragmented and disorganised. At a cosmetic level this can be observed as a reddening and/or thickening of the skin resulting a leathery appearance, skin fragility and irregular pigmentation, loss of tone and elasticity, as well as wrinkling, dryness, sunspots and deep furrow formation.

The term “warts” in the context of the present invention is intended to human papilloma virus (HPV) infections leading to formation of warts on the body, such as the skin, genitals and mouth.

The present composition may also be effective at reducing or minimizing scar tissue or improving cosmesis or functional outcome in a wound and scar reduction, wherein the wound is cutaneous, chronic or for example diabetes associated, and includes cuts and lacerations, surgical incisions, punctures, graces, scratches, compression wounds, abrasions, friction wounds, chronic wounds, ulcers, thermal effect wounds, chemical wounds, wounds resulting from pathogenic infections, skin graft/transplant donor and recipient sites, immune response conditions, oral wounds, stomach or intestinal wounds, damaged cartilage or bone, amputation sides and corneal lesions.

The potency of a composition of the invention may be tested in a model where test compositions (20 μL) are applied topically, once daily, on a 2 cm² area on each flank of anaesthetized CRL:CD(SD)-HR-CD male rats (12 weeks old). 6 different animals are treated with each formulation. Animals are allowed to recover from anesthesia after 2 hours. Dosing with formulations may vary from a single application to several, once daily applications. A visual scoring on erythema, oedema, ulceration and telangiectasia is performed 24 hours after each application. 24 hours after the last application, animals are euthanized by asphyxiation in CO₂ and two 5 mm punch biopsies are taken from each treatment site: one biopsy is snap frozen in liquid nitrogen and stored at −20° C. until analysis for the chemokine KC(CXCL1), the other sample is fixated in formalin at room temperature for histological analysis. Clinical scoring, KC production and histological evaluation of the epidermal and dermal compartment are compared against Picato gel formulation in order to identify new formulations with the same ability to create a strong, local skin reaction, increase KC production and induce necrosis of epidermis and dermis. An increase in any of these parameters is interpreted as an increased potency of the formulation.

In a specific embodiment the composition comprises

0.0001-0.5% by weight ingenol-3-angelate
5-15% by weight polyoxypropylene-15-stearyl ether
0.5-1.5% by weight benzyl alcohol
5-9% by weight fumed silica
80-90% by weight liquid paraffin

The invention is described in further detail in the following examples which are not in any way intended to limit the scope of the invention as claimed.

### EXAMPLES

### Anhydrous Lipid Gel Compositions

### Composition 1

0.05% ingenol-3-angelate
0.9% benzyl alcohol
10% polyoxypropylene-15-stearyl ether
84.05% liquid paraffin

### 5% Aerosil 200P

### Composition 2

0.05% ingenol-3-angelate
0.9% benzyl alcohol
10% polyoxypropylene-15-stearyl ether
20% isopropanol
64.05% liquid paraffin
5% Aerosil 200P (amorphous anhydrous colloidal silicon dioxide)
Compositions 1 and 2 were prepared by dissolving the ingenol-3-angelate in benzyl alcohol and adding polyoxypropylene-15-stearyl ether and, in Composition 2, isopropanol under stirring. Liquid paraffin was added under homogenisation. Finally, fumed silica was added under homogenisation.

### Composition 3

ingenol-3-angelate 0.5 mg/g
Benzyl Alcohol 9 mg/g
Arlamol E 100 mg/g
Paraffin, liquid 840.5 mg/g
Aerosil 200P (amorphous anhydrous colloidal silicon dioxide) 50 mg/g

### Composition 4

ingenol-3-angelate 0.5 mg/g
Benzyl Alcohol 9 mg/g
Arlamol E 100 mg/g
Isopropanol 200 mg/g
Paraffin, liquid 640.5 mg/g
Aerosil 200P (amorphous anhydrous colloidal silicon dioxide) 50 mg/g

### Composition 5

ingenol-3-angelate 0.5 mg/g
Benzyl Alcohol 9 mg/g
Arlamol E (PPG-15 Stearyl Ether) 100 mg/g
Paraffin, liquid 820.5 mg/g
Aerosil 200P (amorphous anhydrous colloidal silicon dioxide) 70 mg/g

### Composition 6

ingenol-3-angelate 0.5 mg/g
Benzyl Alcohol 9 mg/g
Arlamol E (PPG-15 Stearyl Ether) 100 mg/g
Paraffin, liquid 820.5 mg/g
Aerosil R972P 70 mg/g

### Composition 7

ingenol-3-angelate 0.5 mg/g
Benzyl Alcohol 9 mg/g
Arlamol E (PPG-15 Stearyl Ether) 100 mg/g
Paraffin, liquid 800.5 mg/g
Aerosil R972P 90 mg/g

### Composition 8

ingenol-3-angelate 0.5 mg/g
Benzyl Alcohol 9 mg/g
Arlamol E (PPG-15 Stearyl Ether) 100 mg/g
Isopropanol 200 mg/g
Paraffin, liquid 600.5 mg/g
Aerosil R972P 90 mg/g

### Composition 9

ingenol-3-angelate 0.5 mg/g
Dow Corning® ST-Elastomer 10 799.5 mg/g
Dow Corning® ST cyclomethicone 95 mg/g
Isopropyl myristate 95 mg/g
Benzyl alcohol 10 mg/g

### Composition 10

ingenol-3-angelate 0.5 mg/g
Dow Corning® BY 11-030 196 mg/g
Dow Corning® ST cyclomethicone 5 675.5 mg/g
Ethanol 98 mg/g
Benzyl alcohol 10 mg/g
Citrate buffer pH 3.0 20 mg/g

### Composition 11

ingenol-3-angelate 0.5 mg/g
Benzyl alcohol 9 mg/g
Paraffin, liquid 870.5 mg/g
i-hexadecane (Arlamol HD) 50 mg/g
Aerosil P200 (amorphous anhydrous colloidal silicon dioxide) 70 mg/g

### Composition 12

ingenol-3-angelate 0.5 mg/g
Arlamol E (PPG-15 Stearyl Ether) 100 mg/g
Paraffin, liquid 779.5 mg/g
i-hexadecane (Arlamol HD) 50 mg/g
Aerosil P200 (amorphous anhydrous colloidal silicon dioxide) 70 mg/g

### Composition 13

ingenol-3-angelate 0.5 mg/g
Oleyl alcohol 50 mg/g
Paraffin, liquid 829.5 mg/g
i-hexadecane (Arlamol HD) 50 mg/g
Aerosil P200 (amorphous anhydrous colloidal silicon dioxide) 70 mg/g

### Composition 14

ingenol-3-angelate 0.5 mg/g
Diisopropyladipate 100 mg/g
Paraffin, liquid 829.5 mg/g
Aerosil P200 (amorphous anhydrous colloidal silicon dioxide) 70 mg/g

### Composition 15

ingenol-3-angelate 0.5 mg/g
Ethyloleate 100 mg/g
Paraffin, liquid 829.5 mg/g
Aerosil P200 (amorphous anhydrous colloidal silicon dioxide) 70 mg/g

### Composition 16

ingenol-3-angelate 0.5 mg/g
Caprylic/capric triglyceride 50 mg/g
Azone 10 mg/g
Paraffin, liquid 869.5 mg/g
Aerosil P200 (amorphous anhydrous colloidal silicon dioxide) 70 mg/g

### Composition 17

ingenol-3-angelate 0.5 mg/g
Caprylic/capric triglyceride 50 mg/g
NMP 10 mg/g
Paraffin, liquid 860.5 mg/g
Aerosil P200 (amorphous anhydrous colloidal silicon dioxide) 70 mg/g

### Composition 18

ingenol-3-angelate 0.5 mg/g
Caprylic/capric triglyceride 50 mg/g
Ethyl acetate 10 mg/g
Paraffin, liquid 869.5 mg/g
Aerosil P200 (amorphous anhydrous colloidal silicon dioxide) 70 mg/g

### Composition 19

ingenol-3-angelate 0.5 mg/g
Caprylic/capric triglyceride 50 mg/g
Propylenecarbonate 10 mg/g
Paraffin, liquid 869.5 mg/g
Aerosil P200 (amorphous anhydrous colloidal silicon dioxide) 70 mg/g

### Composition 20

ingenol-3-angelate 0.5 mg/g
Caprylic/capric triglyceride 50 mg/g
Eugenol 10 mg·g
Paraffin, liquid 869.5 mg/g
Aerosil P200 (amorphous anhydrous colloidal silicon dioxide) 70 mg/g

### Composition 21

ingenol-3-angelate 0.5 mg/g
Arlamol E (PPG-15 Stearyl Ether) 100 mg/g
Paraffin, liquid 699.5 mg/g
Microcrystalline wax (80° C.) 200 mg/g

### Composition 22

ingenol-3-angelate 0.5 mg/g
Arlamol E (PPG-15 Stearyl Ether) 100 mg/g
Paraffin, liquid 649.5 mg/g
Oleic acid 50 mg/g
Microcrystalline wax (80° C.) 200 mg/g

### Composition 23

ingenol-3-angelate 0.5 mg/g
Dow Corning® ST cyclomethicone 5-NF 90 mg/g
Benzyl alcohol 10 mg
Dow Corning® Silky Wax 100 mg/g
Dow Corning® ST-Elastomer 10 799.5 mg/g

### Composition 24

ingenol-3-angelate 0.5 mg/g
Arlamol E (PPG-15 Stearyl Ether) 100 mg/g
Paraffin, liquid 849.5 mg/g
i-hexadecane 50 mg/g
Cholesterol (100° C.) 50 mg/g

### Composition 25

ingenol-3-angelate 0.5 mg/g
Benzyl alcohol 10 mg/g
Propylene glycol dipelargonate 100 mg/g
Isostearyl isostearate 100 mg/g
Paraffin, liquid 739.5 mg/g
Hydrogenated castor oil 20 mg/g
Cetostearyl alcohol (60° C.) 30 mg/g

### Composition 26

ingenol-3-angelate 0.5 mg/g
Benzyl alcohol 10 mg/g
Propylene glycol dipelargonate 100 mg/g
Isopropyl myristate 100 mg/g
Paraffin, liquid 739.5 mg/g
Hydrogenated castor oil 20 mg/g
Cetostearyl alcohol (60° C.) 30 mg/g

### Composition 27

ingenol-3-angelate 0.5 mg/g
Arlamol E (PPG-15 Stearyl Ether) 100 mg/g
Paraffin, liquid 849.5 mg/g
Hydrogenated castor oil 20 mg/g
Cetostearyl alcohol (60° C.) 30 mg/g

### Composition 28

ingenol-3-angelate 0.5 mg/g
Arlamol E (PPG-15 Stearyl Ether) 120 mg/g
Paraffin, liquid 859.5 mg/g
Hydrogenated castor oil 20 mg/g

### Composition 29

ingenol-3-angelate 0.5 mg/g
Arlamol E (PPG-15 Stearyl Ether) 160 mg/g
Paraffin, liquid 789.5 mg/g
Hydrogenated castor oil 30 mg/g
Aluminium stearate (Melt not higher than 125° C.) 20 mg/g

### Composition 30

ingenol-3-angelate 0.5 mg/g
Arlamol E (PPG-15 Stearyl Ether) 160 mg/g
Oleic acid 50 mg/g
Paraffin, liquid 739.5 mg/g
Hydrogenated castor oil 30 mg/g
Aluminium stearate (Melt not higher than 125° C.) 20 mg/g

### Composition 31

ingenol-3-angelate 0.5 mg/g
Benzyl alcohol 10 mg/g
Paraffin, liquid 939.5 mg/g
Cholesterol (100° C.) 50 mg/g

### Composition 32

ingenol-3-angelate 0.5 mg/g
Benzyl alcohol 10 mg/g
Isopropyl myristate 100 mg/g
Paraffin, liquid 839.5
Hydrogenated castor oil 20 mg/g
Cetostearyl alcohol (60° C.) 30 mg/g

### Composition 33

ingenol-3-angelate 0.5 mg/g
Benzyl alcohol 10 mg/g
Caprylic/capric triglyceride 100 mg/g
Isopropyl myristate 100 mg/g
Paraffin, liquid 739.5
Hydrogenated castor oil 20 mg/g
Cetostearyl alcohol (60° C.) 30 mg/g

### Composition 34

ingenol-3-angelate 0.5 mg/g
Dow Corning® ST-Elastomer 10 789.5 mg/g
Dow Corning® ST cyclomethicone 95 mg/g
Isopropyl myristate 95 mg/g
Benzyl alcohol 10 mg/g
α-tocopherol 10 mg/g

### Composition 35

ingenol-3-angelate 0.5 mg/g
Dow Corning® ST-Elastomer 10 789.5 mg/g
Dow Corning® ST cyclomethicone 95 mg/g
Isopropyl myristate 95 mg/g
Benzyl alcohol 10 mg/g
α-tocopherol 1 mg/g

### Composition 36

ingenol-3-angelate 0.5 mg/g
Dow Corning® ST-Elastomer 10 799.4 mg/g
Dow Corning® ST cyclomethicone 95 mg/g
Isopropyl myristate 95 mg/g
Benzyl alcohol 10 mg/g
α-tocopherol 0.1 mg/g

The compositions were tested for chemical stability by extracting ingenol-3-angelate from the composition by dissolution in a solvent mixture of acetonitrile and phosphoric acid. Identification, assay and determination of organic impurities were determined by reversed phase HPLC with UV detection at 220 nm. Compositions 1-9, 11-14, 16-18 and 20-36 were found to be stable after 3 months at 40° C., indicating that the compositions are likely to have a shelf life for about 2 years at room temperature.

### Example 2

### Results of Skin Penetration Studies

To investigate the skin penetration and permeation of ingenol-3-angelate from compositions of the invention, a skin diffusion experiment was conducted. Full thickness skin from pig ears was used in the study. The ears were kept frozen at −18° C. before use. On the day prior to the experiment the ears were placed in a refrigerator (5±3° C.) for slow defrosting. On the day of the experiment, the hairs were removed using a veterinary hair trimmer. The skin was cleaned for subcutaneous fat using a scalpel and two pieces of skin were cut from each ear and mounted on Franz diffusion cells in a balanced order.

Static Franz-type diffusion cells with an available diffusion area of 3.14 cm² and receptor volumes ranging from 8.6 to 11.1 ml were used in substantially the manner described by T. J. Franz, “The finite dose technique as a valid in vitro model for the study of percutaneous absorption in man”, in Current Problems in Dermatology, 1978, J. W. H. Mall (Ed.), Karger, Basel, pp. 58-68. The specific volume was measured and registered for each cell. A magnetic bar was placed in the receptor compartment of each cell. After mounting the skin, physiological saline (35° C.) was filled into each receptor chamber for hydration of the skin. The cells were placed in a thermally controlled water bath which was placed on a magnetic stirrer set at 400 rpm. The circulating water in the water baths was kept at 35±1° C. resulting in a temperature of about 32° C. on the skin surface. After one hour the saline was replaced by receptor medium, 0.04 M isotonic phosphate buffer, pH 7.4 (35° C.), containing 4% bovine serum albumin. Sink conditions were maintained at all times during the period of the study, i.e. the concentration of the active compounds in the receptor medium was below 10% of the solubility of the compounds in the medium.

The in vitro skin permeation of Compositions 1 to 36 and a hydrogel formulation according to WO 2007/068963 was tested in 6 replicates (i.e. n=6). Each test composition was applied to the skin membrane at 0 hours in an intended dose of 4 mg/cm². A glass spatula was used for the application, and the residual amount of the composition was determined so as to give the amount of the composition actually applied on the skin.

The skin penetration experiment was allowed to proceed for 21 hours. Samples were then collected from the following compartments:

The stratum corneum was collected by tape stripping 10 times using D-Squame® tape (diameter 22 mm, CuDerm Corp., Dallas, Tex., USA). Each tape strip is applied to the test area using a standard pressure for 5 seconds and removed from the test area in one gentle, continuous move. For each repeated strop, the direction of tearing off was varied. The viable epidermis and dermis was then sampled from the skin in a similar fashion.

Samples (1 ml) of the receptor fluid remaining in the diffusion cell were collected and analysed.

The concentration of ingenol-3-angelate in the samples were determined by LC mass spectrometry.

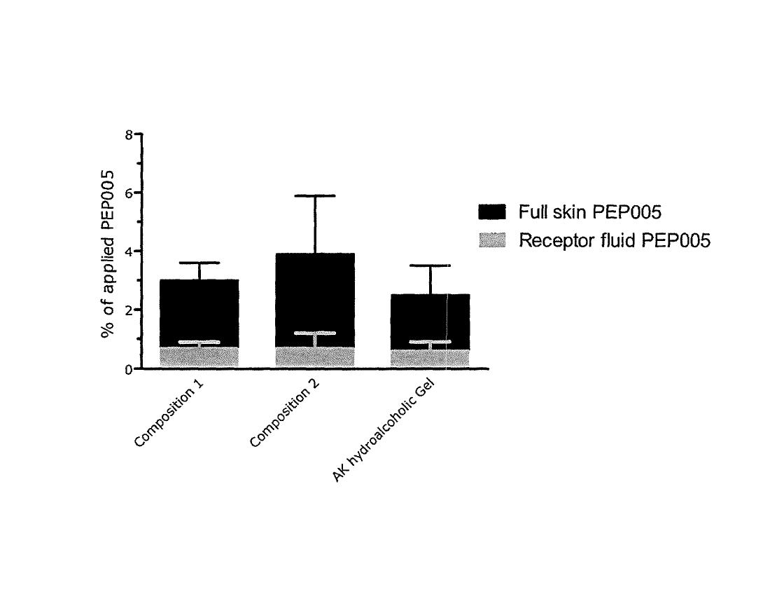

The results appear from FIG. 1 below which shows the amount of ingenol-3-angelate found in viable skin (dermis and epidermis) and receptor fluid in % of the applied dose of Compositions 1 and 2 compared to the hydrogel formulation disclosed in WO 2007/068963. It appears from the FIGURE that the amount of ingenol-3-angelate found in viable skin after application of Compositions 1 and 2 is significantly higher than the amount found in viable skin after application of the hydrogen formulation. On the other hand, very little of the applied ingenol-3-angelate was found in the receptor fluid, suggesting that on application of the present compositions in vivo, only a minor amount of the active ingredient will permeate through the skin into the systemic circulation, thus minimizing the risk of systemic adverse effects.

1. A substantially anhydrous topical composition comprising a homogenous mixture of
(a) a therapeutically effective amount of an ingenol derivative in dissolved form;
(b) an oily solvent for the ingenol derivative,
(c) an acidic compound, and
(d) optionally a pharmaceutically acceptable non-solvent lipid carrier.

2. A composition according to claim 1, wherein the ingenol derivative is selected from the group consisting of ingenol-3-angelate, ingenol-5-angelate, ingenol-20-angelate, 20-O-acetyl-ingenol-3-angelate and 20-deoxy-ingenol-3-angelate.

3. A composition according to claim 2, wherein the ingenol derivative is ingenol-3-angelate.

4. A composition according to claim 1, wherein the oily solvent is selected from the group consisting of vegetable oils, e.g. sesame oil, corn oil, sunflower oil, safflower oil, olive oil, almond oil, avocado oil, jojoba oil, palm kernel oil canola oil, coconut oil, cottonseed oil, peanut oil, soybean oil, wheat germ oil or grape kernel oil, highly purified vegetable oils, e.g. medium chain triglycerides or long chain triglycerides such as castor oil, or synthetic oils such as fatty alcohol esters of C₁₀₋₁₈ alkanoic or alkenoic acids, e.g. isopropyl myristate, isopropyl palmitate, isopropyl isostearate, isopropyl linoleate or isopropyl monooleate, propylene glycol esters, e.g. propylene glycol dipelargonate, polyoxypropylene fatty alkyl ethers, e.g. polyoxypropylene-15-stearyl ether, an alkyl or dialkyl ester such as ethyl oleate, diisopropyl adipate or cetearyl octanoate, a mono- or diglyceride such as glyceryl monooleate, or a fatty alcohol such as oleyl alcohol, or mixtures thereof.

5. A composition according to claim 4, wherein the oily solvent comprises medium chain triglycerides, polyoxypropylene-15-stearyl ether, a volatile silicone oil or a vegetable oil, or a mixture thereof.

6. A composition according to claim 1 further comprising a co-solvent selected from the group consisting of a lower alcohol, e.g. n-propanol, isopropanol, n-butanol, 2-butanol or benzyl alcohol, or a diol, e.g. propylene glycol, or a penetration enhancer selected from the group consisting of an azone, a pyrrolidone, e.g. N-methylpyrrolidone or N-hydroxyalkylpyrrolidone, Transcutol, propylene carbonate, menthol, eucalyptol, nicotinamide, glycerol, mono-di- or polyglycols, ethylacetate or Eugenol.

7. A composition according to claim 6, wherein the acidic compound is selected from the group consisting of fumed silica, fatty acids such as oleic acid, linoleic acid, stearic acid, palmitic acid, lauric acid, capric acid, pelargonic acid, caprylic acid, adipic acid, sebacic acid or enanthic acid.

8. A composition according to claim 1, wherein said oily solvent is included in the composition in an amount in the range of from about 1% to about 98% by weight of the composition, preferably about 2.5-75% by weight, about 5-50% by weight or about 5-40% by weight, such as about 5% or about 10% or about 15% or about 20% or about 25% or about 30% by weight of the composition.

9. A composition according to claim 1, wherein the non-solvent lipid carrier is selected from a mineral oil, e.g. liquid paraffin, or a paraffin selected from paraffins consisting of hydrocarbons with chain lengths from C₅ to C₆₀/the chain lengths peaking at C₁₄₋₁₆, C₁₈₋₂₂/C₂₀₋₂₂/C₂₀₋₂₆, C₂₈₋₄₀, and C₄₀₋₄₄, or mixtures thereof, or an iso-paraffin such as isohexadecane or squalane, or a silicone oil, such as cyclomethicone or dimethicone.

10. A composition according to claim 1, further comprising a viscosity-increasing ingredient.

11. A composition according to claim 10, wherein the viscosity-increasing ingredient is a wax, e.g. microcrystalline wax, silicone wax or hydrogenated castor oil, fumed silica, aluminium stearate, a sterol such as cholesterol, a long-chain saturated fatty alcohol such as cetostearyl alcohol or a silicone rubber or wax such as Dow Corning ST-Elastomer 10 or Dow Corning Silky Wax, or SEPINEO P600 or SEPINEO SE68.

12. A composition according to claim 1 comprising about 0.001-0.5% by weight of the ingenol derivative.

13. A composition according to claim 1 comprising
0.0001-0.5% by weight ingenol-3-angelate
5-15% by weight polyoxypropylene-15-stearyl ether
0.5-1.5% by weight benzyl alcohol
5-9% by weight fumed silica
80-90% by weight liquid paraffin.

14. A composition according to claim 1 for use in the treatment of dermatological diseases or conditions.

15. A composition according to claim 14, wherein the dermatological disease or condition is selected from the group consisting of actinic keratosis, basal cell carcinoma, squamous cell carcinoma, warts, keloids, scars, photoaged or photodamaged skin, or acne.

16. A composition according to claim 2, wherein the oily solvent is selected from the group consisting of vegetable oils, e.g. sesame oil, corn oil, sunflower oil, safflower oil, olive oil, almond oil, avocado oil, jojoba oil, palm kernel oil canola oil, coconut oil, cottonseed oil, peanut oil, soybean oil, wheat germ oil or grape kernel oil, highly purified vegetable oils, e.g. medium chain triglycerides or long chain triglycerides such as castor oil, or synthetic oils such as fatty alcohol esters of C₁₀₋₁₈ alkanoic or alkenoic acids, e.g. isopropyl myristate, isopropyl palmitate, isopropyl isostearate, isopropyl linoleate or isopropyl monooleate, propylene glycol esters, e.g. propylene glycol dipelargonate, polyoxypropylene fatty alkyl ethers, e.g. polyoxypropylene-15-stearyl ether, an alkyl or dialkyl ester such as ethyl oleate, diisopropyl adipate or cetearyl octanoate, a mono- or diglyceride such as glyceryl monooleate, or a fatty alcohol such as oleyl alcohol, or mixtures thereof.

17. A composition according to claim 3, wherein the oily solvent is selected from the group consisting of vegetable oils, e.g. sesame oil, corn oil, sunflower oil, safflower oil, olive oil, almond oil, avocado oil, jojoba oil, palm kernel oil canola oil, coconut oil, cottonseed oil, peanut oil, soybean oil, wheat germ oil or grape kernel oil, highly purified vegetable oils, e.g. medium chain triglycerides or long chain triglycerides such as castor oil, or synthetic oils such as fatty alcohol esters of C₁₀₋₁₈ alkanoic or alkenoic acids, e.g. isopropyl myristate, isopropyl palmitate, isopropyl isostearate, isopropyl linoleate or isopropyl monooleate, propylene glycol esters, e.g. propylene glycol dipelargonate, polyoxypropylene fatty alkyl ethers, e.g. polyoxypropylene-15-stearyl ether, an alkyl or dialkyl ester such as ethyl oleate, diisopropyl adipate or cetearyl octanoate, a mono- or diglyceride such as glyceryl monooleate, or a fatty alcohol such as oleyl alcohol, or mixtures thereof.

18. A composition according to claim 2 further comprising a co-solvent selected from the group consisting of a lower alcohol, e.g. n-propanol, isopropanol, n-butanol, 2-butanol or benzyl alcohol, or a diol, e.g. propylene glycol, or a penetration enhancer selected from the group consisting of an azone, a pyrrolidone, e.g. N-methylpyrrolidone or N-hydroxyalkylpyrrolidone, Transcutol, propylene carbonate, menthol, eucalyptol, nicotinamide, glycerol, mono-di- or polyglycols, ethylacetate or Eugenol.

19. A composition according to claim 3 further comprising a co-solvent selected from the group consisting of a lower alcohol, e.g. n-propanol, isopropanol, n-butanol, 2-butanol or benzyl alcohol, or a diol, e.g. propylene glycol, or a penetration enhancer selected from the group consisting of an azone, a pyrrolidone, e.g. N-methylpyrrolidone or N-hydroxyalkylpyrrolidone, Transcutol, propylene carbonate, menthol, eucalyptol, nicotinamide, glycerol, mono-di- or polyglycols, ethylacetate or Eugenol.

20. A composition according to claim 4 further comprising a co-solvent selected from the group consisting of a lower alcohol, e.g. n-propanol, isopropanol, n-butanol, 2-butanol or benzyl alcohol, or a diol, e.g. propylene glycol, or a penetration enhancer selected from the group consisting of an azone, a pyrrolidone, e.g. N-methylpyrrolidone or N-hydroxyalkylpyrrolidone, Transcutol, propylene carbonate, menthol, eucalyptol, nicotinamide, glycerol, mono-di- or polyglycols, ethylacetate or Eugenol.
